(19) United States
(12) Patent Application Publication
(10) Pub. No.: US 2023/0042633 A1
(43) Pub. Date: Feb. 9, 2023

(54) A SYSTEM AND A METHOD TO ACCURATELY DETERMINE THE CALORIE CONSUMED DURING DAILY ACTIVITIES/EXERCISE

(71) Applicant: Mohan DEWAN, Pune (IN)

(72) Inventor: Mohan DEWAN, Pune (IN)

(21) Appl. No.: 17/793,061

(22) PCT Filed: Feb. 3, 2021
(86) PCT No.: PCT/IB2021/050857

§ 371 Date: Jul. 15, 2022

(30) Foreign Application Priority Data
Apr. 8, 2020 (IN) 202021015364

(51) Int. Cl. A63B 24/00; G16H 20/30

(52) U.S. Cl. CPC A63B 24/0062; G16H 20/30; A63B 2230/75

(57) ABSTRACT

The present disclosure discloses a system (100) to accurately determine the calorie consumed during daily activities/exer cise of a user associated with a user device (102) having a plurality of sensors. The system (100) comprises a rules repository (104), an input module (106), a user repository (108), a monitoring module (110), a first analysis module (112), a second analysis module (114), a third analysis module (116) and a calorie consumption module (118). The rules repository (104) stores three sets of pre-determined calculating rules and analysis rules. The input module (106) enables user to enter a plurality of primary user details and store it in the user repository (108). The monitoring module (110) receive a plurality of dynamic user data, a surroundings data and a user activity data using the sensors. The received data along with the primary user details is analysed. A first result, a second result and a third result are calculated post analysis and are added to get a final calorie result.

### FIELD

The present disclosure relates to the field of a system and method to accurately determine the calorie consumed during daily activities/exercise.

### Definitions

As used in the present disclosure, the following terms are generally intended to have the meaning as set forth below, except to the extent that the context in which they are used indicate otherwise.

User—The term “user” hereinafter refers to a person who uses the system of the present disclosure of the system and method to accurately determine the calorie consumed during daily activities/exercise.

### BACKGROUND

The background information herein below relates to the present disclosure but is not necessarily prior art.

Today, there are many fitness tracking applications and systems available in smart electronic devices which track the calories lost during a session. Often the system misses out crucial parameters in judging the calorie count. One of the parameters is temperature of the body and the environment where the person is working out. If the environment temperature is less than the body temperature, energy consumption is high and vice versa. Second parameter could be steps taken by the individual against the gravity, as it takes more effort and calories to go against the gravity. More the angle of inclination of the steps, more the calories consumed. Last parameter is the weight of the person. The daily weight differs for each individual and is different at different time of the day. Further, the calories consumed also depend on the distance travelled by the person, for example on the treadmill. Presently, there is no system or method that takes all the afore-mentioned parameters into account for determining the calory consumption of an individual.

There is, therefore, a need for a system and method to accurately determine the calorie consumed during daily activities/exercise that alleviates the aforementioned drawbacks.

### OBJECTS

Some of the objects of the present disclosure, which at least one embodiment herein satisfies, are as follows:

It is an object of the present disclosure to ameliorate one or more problems of the prior art or to at least provide a useful alternative.

An object of the present disclosure is to provide a system to accurately determine the calorie consumed during daily activities/exercise.

Another object of the present disclosure is to provide a system that considers the parameters of body and environment temperature in calculating the calories consumed.

Still another object of the present disclosure is to provide a system that considers the weight of the person at different times of the day for calculating calorie consumption.

Yet another object of the present disclosure is to provide a system that considers the angle of inclination and gravity while calculating the calories consumed.

Still another object of the present disclosure is to provide a system that improves the accuracy of calculation of the calories consumed.

Yet another object of the present disclosure is to provide a system that considers the intensity of the activity performed by the user to correctly calculate the calories consumed by the user.

Other objects and advantages of the present disclosure will be more apparent from the following description, which is not intended to limit the scope of the present disclosure.

### SUMMARY

The present disclosure envisages a system to accurately determine the calorie consumed during daily activities/exercise.

The system to accurately determine the calorie consumed during daily activities/exercise of a user, wherein the user is associated with a user device and said user device having a plurality of sensors are installed in it.

The system comprises a rules repository, an input module, a user repository, a monitoring module, a first analysis module, a second analysis module, a third analysis module and a calorie consumption module.

The rules repository is configured to store a set of first pre-determined analysis rule, a set of second pre-determined analysis rule, a set of third pre-determined analysis rules, a set of first pre-determined calculating rules, a set of second pre-determined calculating rules and a set of third pre-determined calculating rules.

The input module configured to receive a plurality of primary user details from the user associated via the user device associated with the user.

In an embodiment, the primary user details are selected from a group of name, gender, height, present weight and time of inputting weight.

The user repository is configured to cooperate with the input module to receive the primary user details and further configured to store the primary user details.

The monitoring module is configured to cooperate with said sensors to receive a plurality of dynamic user data, a plurality of surroundings data and a plurality of user activity data via user device.

In an embodiment, the dynamic user data is measured body temperature of the user.

In an embodiment, the surroundings data are selected from a group of ambient temperature, gravity and incline of the surface on which the user is doing the activity.

In an embodiment, the user activity data are selected from a group of running speed, steps taken by the user, distance covered by the user.

The first analysis module is configured to cooperate with the monitoring module, the user repository and the rules repository to receive the set of first pre-determined analysis rules, the set of first pre-determined the calculating rules, the primary user details and the user activity data and is further configured to analyse the primary user details and the user activity data based on the corresponding analysis rules and is further configured to calculate a first result using the analysed primary user details and the analysed user activity data and the set of first pre-determined calculating rules.

The second analysis module is configured to cooperate with the monitoring module and the rules repository to receive the set of second pre-determined analysis rules, the set of second pre-determined calculating rules and the user activity data and is further configured to analyse and bifurcate the user activity data based on intensity by applying the set of second pre-determined analysis rules. The second analysis module is further configured to calculate a second result using on the bifurcated activity data, the user activity data and the primary user details by applying the set of second pre-determined calculating rules.

The third analysis module is configured to cooperate with the monitoring module and the rules repository to receive the surroundings data, the set of third pre-determined calculating rules, the set of third pre-determined analysis rules and the dynamic user data and is further configured to analyse and calculate a third result using the surroundings data, and dynamic user data by applying the set of third pre-determined analysis rules and the set of third pre-determined calculating rules.

The calorie consumption module is configured to cooperate with the first analysis module, the second analysis module, and the third analysis module to receive the first result, the second result and the third result and is further configured to calculate a final calorie result by adding the first result, the second result and the third result.

The present disclosure also envisages a method to accurately determine the calorie consumed during daily activities/exercise.

### BRIEF DESCRIPTION OF THE ACCOMPANYING DRAWING

A system and a method to accurately determine the calorie consumed during daily activities/exercise of the present disclosure will now be described with the help of the accompanying drawing, in which:

FIG. 1 illustrates a system to accurately determine the calorie consumed during daily activities/exercise in accordance with an embodiment of the present disclosure; and

FIGS. 2a to 2d illustrates a flow chart depicting steps involved in a method to accurately determine the calorie consumed during daily activities/exercise.

### LIST OF REFERENCE NUMERALS

100—System
102—application loaded user device
104—rules repository
106—input module
108—user repository
110—monitoring module
112—first analysis module
114—second analysis module
116—third analysis module
118—calorie consumption module

### DETAILED DESCRIPTION

Embodiments, of the present disclosure, will now be described with reference to the accompanying drawing.

Embodiments are provided so as to thoroughly and fully convey the scope of the present disclosure to the person skilled in the art. Numerous details, are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise.

The terms “comprises,” “comprising,” “including,” and “having,” are open ended transitional phrases and therefore specify the presence of stated features, elements, modules, units and/or components, but do not forbid the presence or addition of one or more other features, elements, components, and/or groups thereof. The particular order of steps disclosed in the method and process of the present disclosure is not to be construed as necessarily requiring their performance as described or illustrated. It is also to be understood that additional or alternative steps may be employed.

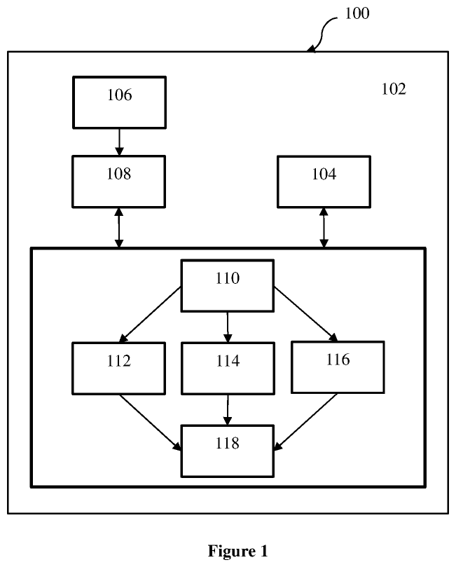

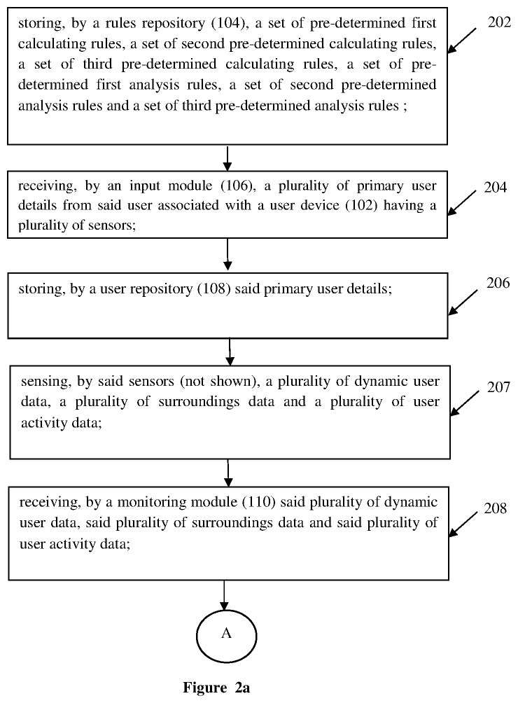

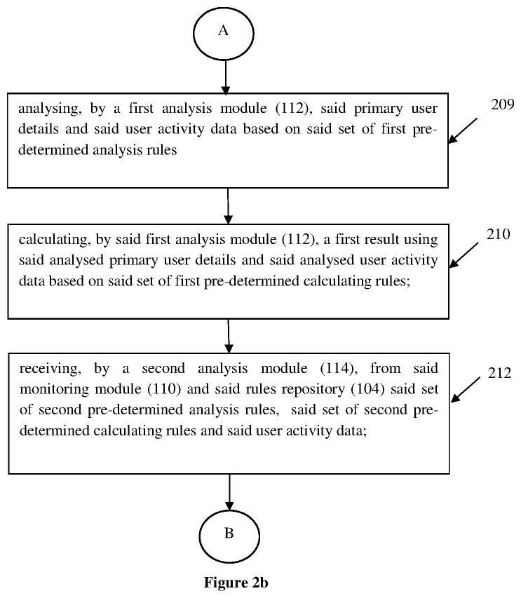

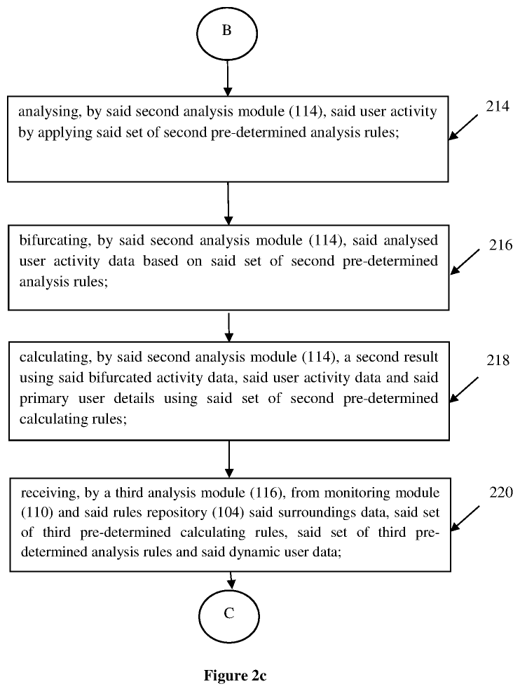

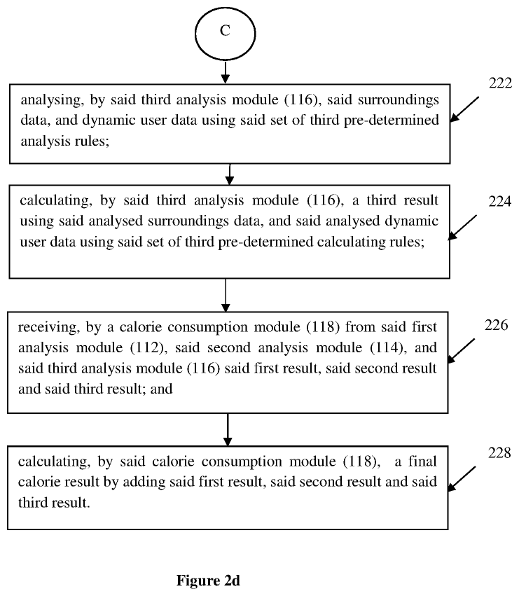

A system (hereinafter referred to as “system 100”) and a method (hereinafter referred to as “method 200”) to accurately determine the calorie consumed during daily activities/exercise, of the present disclosure is now being described with reference to FIG. 1 through FIG. 2d. The system (100) to accurately determine the calorie consumed during daily activities/exercise of a user, wherein the user is associated with a user device (102) and said user device having a plurality of sensors are installed in it.

The system (100) comprises a rules repository (104), an input module (106), a user repository (108), a monitoring module (110), a first analysis module (112), a second analysis module (114), a third analysis module (116) and a calorie consumption module (118).

The rules repository (104) is configured to store a set of first pre-determined analysis rule, a set of second pre-determined analysis rule, a set of third pre-determined analysis rules, a set of first pre-determined calculating rules, a set of second pre-determined calculating rules and a set of third pre-determined calculating rules.

The input module (106) configured to receive a plurality of primary user details via the user device (102) associated with the user.

In an embodiment, the primary user details are selected from a group of name, gender, height, present weight and time of inputting weight.

The user repository (108) is configured to cooperate with the input module to receive the primary user details and further configured to store the primary user details.

The monitoring module (110) is configured cooperate with said sensors to receive a plurality of dynamic user data, a plurality of surroundings data and a plurality of user activity data via the user device (102).

In an embodiment, the dynamic user data is measured body temperature of the user.

In an embodiment, the surroundings data are selected from a group of ambient temperature, gravity and incline of the surface on which the user is doing the activity.

In an embodiment, the user activity data are selected from a group of running speed, steps taken by the user, distance covered by the user.

The first analysis module (112) is configured to cooperate with the monitoring module (110), the user repository (108) and the rules repository (104) to receive the set of first pre-determined analysis rules, the set of first pre-determined calculating rules, the primary user details and the user activity data and is further configured to analyse the primary user details and the user activity data based on said set of first pre-determined analysis rules and further configured to calculate a first result using said analysed primary user details and said analysed user activity data and said set of first pre-determined calculating rules.

The second analysis module (114) is configured to cooperate with the monitoring module (110) and the rules repository (104) to receive the set of second pre-determined analysis rules, the set of second pre-determined calculating rules and the user activity data and is further configured to analyse and bifurcate the user activity data based the set of second pre-determined analysis rules. The second analysis module (114) is further configured to calculate a second result using on the bifurcated activity data, the user activity data and the primary user details by applying the set of second pre-determined calculating rules.

The third analysis module (116) is configured to cooperate with the monitoring module (110) and the rules repository (104) to receive the surroundings data, the set of third pre-determined calculating rules, the set of third pre-determined analysis rules and the dynamic user data and is further configured to analyse and calculate a third result using the analysed surroundings data, and analysed dynamic user data by applying the set of third pre-determined analysis rules and the set of third pre-determined calculating rules.

The calorie consumption module (118) is configured to cooperate with the first analysis module (112), the second analysis module (114), and the third analysis module (116) to receive the first result, the second result and the third result and is further configured to calculate a final calorie result by adding the first result, the second result and the third result.

In an embodiment, the monitoring module (110), the first analysis module (112), the second analysis module (114), the third analysis module (116), the calorie consumption module (118) are implemented using one or more one processor(s).

Referring to FIG. 2a-2d, discloses a method (200) to accurately determine the calorie consumed during daily activities/exercise of a user. The method 200 comprises the following steps:
At step (202), storing (202), using a rules repository (104), a set of pre-determined first calculating rules, a set of second pre-determined calculating rules, a set of third pre-determined calculating rules, a set of pre-determined first analysis rules, a set of second pre-determined analysis rules and a set of third pre-determined analysis rules;
At step (204), receiving (204), using an input module (106), a plurality of primary user details from said user associated with a user device (102) having a plurality of sensors;
At step (206), storing (206), using a user repository (108) said primary user details;
At step (207), sensing (207), using said sensors (not shown), a plurality of dynamic user data, a plurality of surroundings data and a plurality of user activity data;
At step (208), receiving (208), using a monitoring module (110) said plurality of dynamic user data, said plurality of surroundings data and said plurality of user activity data;
At step (209), analysing (209), using a first analysis module (112), said primary user details and said user activity data based on said set of first pre-determined analysis rules;
At step (210), calculating (210), using said first analysis module (112), a first result using said analysed primary user details and said analysed user activity data based on said set of first pre-determined calculating rules;
At step (212), receiving (212), using a second analysis module (114), from said monitoring module (110) and said rules repository (104) said set of second pre-determined analysis rules, said set of second pre-determined calculating rules and said user activity data;
At step (214), analysing (214), using said second analysis module (114), said user activity by applying said set of second pre-determined analysis rules;
At step (216), bifurcating (216), using said second analysis module (114), said analysed user activity data based on said set of second pre-determined analysis rules;
At step (218), calculating (218), using said second analysis module (114), a second result using said bifurcated activity data, said user activity data and said primary user details using said set of second pre-determined calculating rules;
At step (220), receiving (220), using a third analysis module (116), from monitoring module (110) and said rules repository (104) said surroundings data, said set of third pre-determined calculating rules, said set of third pre-determined analysis rules and said dynamic user data;
At step (222), analysing (222), using said third analysis module (116), said surroundings data, and dynamic user data using said set of third pre-determined analysis rules;
At step (224), calculating (224), using said third analysis module (116), a third result using said analysed surroundings data, and said analysed dynamic user data using said set of third pre-determined calculating rules;
At step (226), receiving (226), a calorie consumption module (118) from said first analysis module (112), said second analysis module (114), and said third analysis module (116) said first result, said second result and said third result; and
At step (228), calculating (228), using said calorie consumption module (118), a final calorie result by adding said first result, said second result and said third result.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

### Technical Advancements

The present disclosure described herein above has several technical advantages including, but not limited to, the realization of a system and a method to accurately determine the calorie consumed during daily activities/exercise that:
considers the parameters of body and environment temperature in calculating the calories consumed;
considers the weight of the person at different times of the day for calculating calorie consumption;
considers the angle of inclination and gravity while calculating the calories consumed;
improves the accuracy of calculation of the calories consumed; and
considers the intensity of the activity performed by the user to correctly calculate the calories consumed by the user.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A system (100) to accurately determine the calorie consumed during daily activities/exercise of a user, wherein said user is associated with a user device (102) and said user device having a plurality of sensors are installed in it, said system (100) comprising:
a rules repository (104) configured to store a set of first pre-determined analysis rule, a set of second pre-determined analysis rule, a set of third pre-determined analysis rules, a set of first pre-determined calculating rules, a set of second pre-determined calculating rules and a set of third pre-determined calculating rules;
an input module (106) configured to receive a plurality of primary user details via said user device (102) associated with said user;
a user repository (108) configured to cooperate with said input module to receive said primary user details and further configured to store said primary user details;
a monitoring module (110) configured to cooperate with said sensors to receive a plurality of dynamic user data, a plurality of surroundings data and a plurality of user activity data via user device (102);
a first analysis module (112) configured to cooperate with said monitoring module (110), said user repository (108) and said rules repository (104) to receive said set of first pre-determined analysis rules, said set of first pre-determined said calculating rules, said primary user details and said user activity data and further configured to analyse said primary user details and said user activity data based on said set of first pre-determined analysis rules and further configured to calculate a first result using said analysed primary user details and said analysed user activity data and said set of first pre-determined calculating rules;
a second analysis module (114) configured to cooperate with said monitoring module (110) and said rules repository (104) to receive said set of second pre-determined analysis rules, said set of second pre-determined calculating rules and said user activity data and further configured to analyse and bifurcate said user activity data based on said set of second pre-determined analysis rules and further configured to calculate a second result using said bifurcated activity data, said user activity data and said primary user details by using said set of second pre-determined calculating rules;
a third analysis module (116) configured to cooperate with said monitoring module (110) and said rules repository (104) to receive said surroundings data, said set of third pre-determined calculating rules, said set of third pre-determined analysis rules and said dynamic user data and further configured to analyse and calculate a third result based on said analysed surroundings data and said analysed dynamic user data by using said set of third pre-determined analysis rules and said set of third pre-determined calculating rules; and
a calorie consumption module (118) configured to cooperate with said first analysis module (112), said second analysis module (114), and said third analysis module (116) to receive said first result, said second result and said third result and further configured to calculate a final calorie result by adding said first result, said second result and said third result;
wherein said monitoring module (110), said first analysis module (112), said second analysis module (114), said third analysis module (116), said calorie consumption module (118) are implemented using one or more one processor(s).

2. The system (100) as claimed in claim 1, wherein said primary user details can be selected from a group of name, gender, height, present weight and time of inputting weight.

3. The system (100) as claimed in claim 1, wherein said dynamic user data is body temperature.

4. The system (100) as claimed in claim 1, wherein said surroundings data can be selected from a group of ambient temperature, gravity and incline of the surface on which said user is doing the said activity.

5. The system (100) as claimed in claim 1, wherein said user activity data can be selected from a group of running speed, steps taken by said user, distance covered by said user.

6. A method (200) to accurately determine the calorie consumed during daily activities/exercise of a user, wherein said method (200) comprising steps of:
storing (202), by a rules repository (104), a set of pre-determined first calculating rules, a set of second pre-determined calculating rules, a set of third pre-determined calculating rules, a set of pre-determined first analysis rules, a set of second pre-determined analysis rules and a set of third pre-determined analysis rules;
receiving (204), by an input module (106), a plurality of primary user details from said user associated with a user device (102) having a plurality of sensors;
storing (206), by a user repository (108) said primary user details;
sensing (207), by said sensors (not shown), a plurality of dynamic user data, a plurality of surroundings data and a plurality of user activity data;
receiving (208), by a monitoring module (110) said plurality of dynamic user data, said plurality of surroundings data and said plurality of user activity data;
analysing (209), by a first analysis module (112), said primary user details and said user activity data based on said set of first pre-determined analysis rules;
calculating (210), by said first analysis module (112), a first result using said analysed primary user details and said analysed user activity data based on said set of first pre-determined calculating rules;
receiving (212), by a second analysis module (114), from said monitoring module (110) and said rules repository (104) said set of second pre-determined analysis rules, said set of second pre-determined calculating rules and said user activity data;
analysing (214), by said second analysis module (114), said user activity by applying said set of second pre-determined analysis rules;
bifurcating (216), by said second analysis module (114), said analysed user activity data based on said set of second pre-determined analysis rules;
calculating (218), by said second analysis module (114), a second result using said bifurcated activity data, said user activity data and said primary user details using said set of second pre-determined calculating rules;
receiving (220), by a third analysis module (116), from monitoring module (110) and said rules repository (104) said surroundings data, said set of third pre-determined calculating rules, said set of third pre-determined analysis rules and said dynamic user data;
analysing (222), by said third analysis module (116), said surroundings data, and dynamic user data using said set of third pre-determined analysis rules;
calculating (224), by said third analysis module (116), a third result using said analysed surroundings data, and said analysed dynamic user data using said set of third pre-determined calculating rules;
receiving (226), by a calorie consumption module (118) from said first analysis module (112), said second analysis module (114), and said third analysis module (116) said first result, said second result and said third result; and
calculating (228), by said calorie consumption module (118), a final calorie result by adding said first result, said second result and said third result.
